(19) United States
(12) Patent Application Publication
(10) Pub. No.: US 2007/0053787 A1
(43) Pub. Date: Mar. 8, 2007

(54) Method of evaluating and measuring the smell of urine in a material

(76) Inventors: Marilyn Roberts, Oshkosh, WI (US); Karen Marie Menard, Neenah, WI (US); JoAnn Adele Brooks, Arlington, TX (US); Melanie Kading, Oshkosh, WI (US); Gregory Floyd Keep, Appleton, WI (US); David William Koenig, Menasha, WI (US); Richard Charles Zepp, Menasha, WI (US); Elizabeth Deibler Gadsby, Marietta, GA (US); Jaeho Kim, Roswell, GA (US)

Correspondence Address: KIMBERLY-CLARK WORLDWIDE, INC., 401 NORTH LAKE STREET, NEENAH, WI, 54956, US

(21) Appl. No.: 11/222,710

(22) Filed: Sep. 8, 2005

(51) Int. Cl. A61L 9/00

(52) U.S. Cl. 422/5

(57) ABSTRACT

A method of evaluating and measuring the smell of urine is disclosed. The method includes collecting urine samples from at least two donors and pooling the samples together into a combined sample. At least about 99% of the microbes are then removed from the combined sample to obtain a microbe-free sample. The microbe-free sample is then inoculated with at least two known microbes. The method also includes introducing the inoculated sample into a material or product and allowing the microbes to grow and emit malodors. The material or product is then evaluated for malodors that smell like urine to establish an odor intensity level. The method further includes recording the odor intensity level and scoring the odor intensity level.

### BACKGROUND OF THE INVENTION

Odor is often associated with the use of personal care products which are used to collect and/or limit the migration of body fluids onto external clothing. Disposable absorbent articles such as infant diapers; feminine hygiene products, for example, sanitary napkins, pantyliner and tampons; adult incontinence garments such as briefs, guards and undergarments; bed pads; child training pants, perspiration shields, as well as other personal care products, normally worn adjacent to a human body, can acquire malodors. The smell of urine is common in such articles since many are designed specifically to hold and retain urine. Malodors, which means having a bad odor or being ill-smelling, are negatively perceived by consumers, users and caregivers. Today, more attention is being given by manufacturers of such absorbent articles to mask, disguise, conceal, obscure or eliminate such malodors. Body odors can be transferred from a human body to a personal care product in various forms, including solid, semi-solid, liquid or gaseous form. An absorbent article can come in contact with solid or semi-solid fecal waste material emitted from the body. In addition, an absorbent article can be designed to absorb a certain quantity of a liquid or viscous body fluid, such as urine, menses, perspiration, BM, etc. Furthermore, body odors may be transferred to the absorbent article as vapors or gases.

Reduction of odor formation and potential control of offensive odors in personal care products is now being required by today's consumers, users and caregivers. In addition, the ability to eliminate such malodors can be a source of brand identification for a particular manufacturer. Greater business support is being given to researchers to be able to eliminate malodors and relieve consumers of this embarrassing signal of their physiological condition. There is also greater awareness being given to the special need of evaluating how personal care products actually manage malodors related to urine. Although many consumers, users, and caregivers may not have the ability to detect urine odors, especially as they age, they are concerned about others detecting the odor on or near them. Because of this dampened sense of smell, it can be difficult or extremely expensive to evaluate how products manage malodors through a normal wear study. Thus, it is critical to establish a reliable, representative test method to evaluate urine malodor management, to allow inexpensive screening of materials and products for the ability to manage odors.

Now a method of evaluating and measuring the smell of urine in a material or product has been invented. This method determines which microbes are found in worn, urine-insulted disposable absorbent materials or products. Knowing this, researchers may be able to identify certain chemical compounds that can be used to mask, conceal, prevent or eliminate such malodors.

### SUMMARY OF THE INVENTION

Briefly, this invention relates to a method of evaluating and measuring the smell of urine in a material or product. The method includes the steps of collecting urine samples from at least two donors and pooling the samples together into a combined sample. At least about 99% of the microbes are then removed from the combined sample to obtain a microbe-free sample. The microbe-free sample is then inoculated with at least two known microbes. The method also includes introducing the inoculated sample into a material or product and allowing the microbes to grow and emit malodors. The material or product is then evaluated for malodors that smell like urine to establish an odor intensity level. The method further includes recording the odor intensity level and scoring the odor intensity level.

### DETAILED DESCRIPTION

The current trends in personal and household hygiene have made consumers more aware that offensive odors are socially unacceptable. As a result, there are a variety of products in the marketplace to control malodors such as body odor, halitosis, household odors and pet odor. Accordingly, test methods and devices to detect and measure odor have become more prevalent. A study which employs human olfaction to detect and assess odors is very beneficial to manufacturers of personal care products, especially disposable absorbent articles.

In order to develop materials and/or products that can deliver consumer discernable odor control benefits, the need for evaluating urine and hygiene-related odors has been identified. Although scientific instruments can measure the mass of a gas, the human nose has been identified as one device that can detect and interpret odor. The nose has been shown to be one of the most sensitive and discriminating tools for detecting complex odors, especially at low levels. Kimberly-Clark Corporation, having an office at 401 North Lake Street, Neenah, Wis. 54915, has devised a method of enhancing the smell of urine in a material or product such that it can be evaluated by the human nose. Urine is fluid and dissolved substances secreted by the kidneys, stored in the bladder, and excreted from the body through the urethra. By knowing which microbes can produce a pungent smell identical or similar to urine, one can use the human nose as the primary instrument to establish an objective, quantitative test to determine which compounds or chemical compositions can be used to mask, conceal or eliminate such malodors, especially in personal care products.

The method of evaluating and measuring the smell of urine in a material or product includes collecting urine samples from at least two donors and pooling the samples together into a combined sample. The actual number of urine donors can vary. The maximum number of urine donors can be the entire human population. A reasonable number of urine donors can range from 2 to about 1,000. A more manageable number of urine donors can range from 2 to about 100. Desirably, the number of urine donors will range from between 3 to about 50. More desirably, the number of urine donors can range from between 4 to about 40. Even more desirably, the number of urine donors can range from between 5 to about 30. This method of collecting urine samples allows urine odors to be tested which are realistic, representative of real personal care product usage, and much less expensive than using simulated urine.

It has been found that the best time for collecting urine samples from donors is at their first morning void. People who should be considered as possible urine donors need to be healthy. Males and females can both be urine donors. Desirably, the urine donors will be between the ages of 18 and 70. Younger and/or older urine donors can be used, if desired. Donors who exhibit certain physical criteria should not be used. For example, a person experiencing vaginal or penile secretions, menstruation or infection should not donate urine. In addition, people experiencing a bladder, kidney or urinary tract infection should not donate urine. Also, people currently using insulin should not donate urine. Likewise, people currently using oral or injected antibiotic medication, prescription or otherwise, or who have used such medication in the last seven days should not donate urine. Females who are pregnant or lactating should not donate urine. Furthermore, people who have consumed alcohol or asparagus within 24 hours of the scheduled urine collection time should not donate urine. Still further, people who have consumed coffee within 4 hours prior to bedtime or who have consumed coffee prior to collection of the morning urine specimen should not donate urine. Lastly, people who have used a personal hygiene spray, cream or powder on the genital/anal area since bathing should not donate urine.

It should be noted that the method of this invention does not utilize urine collected from animals, such as dogs, cats, etc., because their urine may smell different from the urine collected from humans. However, if one was inclined to use urine from animals because they contemplated making, using or selling materials and/or products directed to the pet industry, one could certainly do so.

A urine donor should be capable of providing a urine sample each day of testing. The actual test period can vary. Each donor should be provided with a clean, new urine specimen container in which to collect his/her first morning void of urine. The urine specimen can be collected at home or at a designated site, such as a clinical lab. If the urine is collected at home, it should be dropped off in its container at a designated site within a prescribed time period. The test sponsor should identify one or more persons to visually evaluate each urine specimen while it is still in its container. The pH of each urine specimen should also be determined. The pH of a urine specimen can be determined using an indicator strip or other means known to those skilled in the art. Only urine that is clear, free of precipitate and having a yellow, straw or amber color should be used. In addition, only those urine specimens that have a pH of from between 4.5 and 8.0 should be used. All of the urine specimen having a pH outside of this range should not be used. Any urine specimen that does not meet the requirements outlined above should be flushed down a toilet and the specimen container placed in a biohazard bag for proper disposal and/or decontaminated.

The urine specimens are then pooled into a combined sample. The combined sample can be stored in a clean glass vessel large enough to hold all of the urine needed for one day of testing.

It should be noted that the combined sample can be placed on ice and placed in a cooler if it needs to be stored for more than a short period of time. The combined sample is then treated to remove essentially all microbes that are present. By “essentially all” it is meant that at least about 99% of the microbes are removed from the combined sample. Desirably, at least about 99.01% of the microbes present in the combined sample are removed or destroyed. More desirably, about 99.1% to about 99.9% of the microbes present in the combined sample are removed or destroyed. Most desirably, 100% of the microbes present in the combined sample are removed or destroyed. The microbes can be removed by filtering, by sterilizing, by autoclave or by any other means known in the art for removing or killing off the microbes. Once essentially all microbes that are present in the combined sample are removed or killed off, a microbe-free sample is obtained.

Filtering is a common means used to obtain an essentially micro-free sample. Filtering can be accomplished in a laminar flow hood using a sterile membrane filter apparatus and sterile 0.22 micrometer (μm) filters. A filter that works well is a pre-sterilized, cellulose acetate low protein binding membrane sold under the name (Whatman #2). Any other apparatus or equipment, known to those skilled in the art, can be used to filter the combined sample of urine. The filtering step will remove at least about 99% of the microbes present in the combined sample. The micro-free sample is then placed in one or more sterile glass containers. The micro-free sample can be kept refrigerated for up to 24 to 36 hours prior to being inoculated, if need be. Desirably, the micro-free sample will be inoculated within 12 hours.

The method also includes inoculating the micro-free sample of urine with at least two microbes. A microbe is any organism too small to be visible to the naked eye. A microbe is a minute life form known as a microorganism. The micro-free sample of urine can be inoculated with equal or non-equal counts of at least two microbes. Desirably, the micro-free sample of urine is inoculated with equal counts of at least two microbes. More desirably, the micro-free sample of urine is inoculated with at least three microbes. Even more desirably, the micro-free sample of urine is inoculated with at least four microbes. Most desirably, the micro-free sample of urine is inoculated with five or more microbes. The resulting inoculated sample will exhibit a total microbe concentration of about 4×10⁶ Colony Forming Units per millimeter (CFU/ml), which is representative of bacteria counts of worn, urine-insulted absorbent articles. The resulting inoculated sample can exhibit a different total microbe concentration id desired.

This inoculated sample is then introduced into or onto a material or product. By “introduced” it is meant that the inoculated sample is deposited on the surface of a material or product and is allowed to enter, flow into, penetrate or be absorbed into the material or product. Whether the inoculated sample will actually enter, flow into, penetrate or be absorbed into the material or product will depend on the composition and/or construction of the material or product and the presence of any special coating or treatment that may be on the outer surface of the material or product. The microbes in the inoculated sample will be allowed to grow on the material or product that has been insulted with the inoculated urine sample.

The step of removing or destroying the original microbes and then inoculating the material or product with new microbes will assure that any microbes that could pose a safety hazard for those persons evaluating the samples (odor panelists) are not present. Additionally, it standardizes the malodor producing organisms present in the test sample to allow for a reliable and realistic test.

Before addressing the mechanisms that can facilitate growth of the microbes, a description of certain odors and microbes that emit similar odors may be in order. Laboratory testing by trained humans, each exhibiting a keen sense of smell, have identified five malodor attributes according to the primary chemicals that generate an olfactory sensation. Each attribute is also compared to common odors that describe its character. The selection of these malodor attributes is based on patent documents, literature references and internal gas chromatograph data from human urine inoculated with the known microbes: Proteus mirabilis ATCC 7002, Enterococcus faecalis ATCC 2912, Escherichia coli ATCC 11229 and Klebsiella pneumoniae ATCC 10031. “ATCC” stands for American Type Culture Collection and the specific number refers to the particular strain of the microbe.

Five malodor attributes are listed below:
1. Ammonia: household ammonia; diaper pail; pungent; sharp.
2. Isovaleric acid: body odor; limburger cheese; dirty socks; rancid oils, dirty gym bag.
3. Dimethyl disulfide: sulfur; sauerkraut; boiled cabbage; rotten eggs; sewer gas; outhouse; fecal.
4. Trimethylamine: fishy odor; dead fish; rotten meat; dead animals.
5. Isobutyraldehyde: pungent; yeasty; malty odor.

By introducing the inoculated urine sample, containing two or more known microbes, into or onto a material or product and allowing the microbes to grow and multiply, one can enhance the amount of malodors emitted from the material or product. The particular microbes that are introduced into or onto the material or product will emit malodors that are similar to or are the same as malodors customarily associated with the smell of urine. By experimentation, it has been discovered that certain microbes need to be used and each will emit specific odors. In addition, certain conditions, such as time, temperature, absence of light, etc., have to be taken into consideration in order to maximize the growth of the particular known microbes.

It has also been discovered that at least two known microbes should be inoculated into the filtered urine sample. Desirably, one of the two known microbes should be Proteus mirabilis ATCC 7002 or a genetically engineered bacterium that has similar metabolic enzymes as Proteus mirabilis ATCC 7002, but is a different organism. Furthermore, at least three known microbes can be inoculated into the filtered urine sample and two of the three known microbes should include Proteus mirabilis ATCC 7002 and Enterococcus faecalis ATCC 2912 or two genetically engineered bacterium that have similar metabolic enzymes as Proteus mirabilis ATCC 7002 and Enterococcus faecalis ATCC 2912, but are different organisms. Still further, at least four known microbes can be inoculated into the filtered urine sample and three of the four known microbes should include Proteus mirabilis ATCC 7002, Enterococcus faecalis ATCC 2912 and Escherichia coli ATCC 11229 or three genetically engineered bacterium that have similar metabolic enzymes as Proteus mirabilis ATCC 7002, Enterococcus faecalis ATCC 2912 and Escherichia coli ATCC 11229, but are different organisms. Most desirably, the filtered urine sample is inoculated with the following four known microbes: Proteus mirabilis ATCC 7002, Enterococcus faecalis ATCC 2912, Escherichia coli ATCC 11229 and Klebsiella pneumoniae ATCC 10031 or four genetically engineered bacterium that have similar metabolic enzymes as Proteus mirabilis ATCC 7002, Enterococcus faecalis ATCC 2912, Escherichia coli ATCC 11229, and Klebsiella pneumoniae ATCC 10031, but are different organisms.

Additional microbes can be inoculated into the micro-free sample, if desired. Some other microbes that can be used include but are not limited to: Cornebacterium ammoniagenes ATCC 6871; Lactobacillus acidophilus ATCC 314; Staphylococcus epidermidis ATCC 146; Streptococcus mitis ATCC 6249; and Streptocuccus pneumoniae (alpha-hemolytic) ATCC 6301 or a genetically engineered bacterium that has similar metabolic enzymes as one of these microbes, but is a different organism.

Returning to the preparation of the known microbes, it has been found that the inoculated urine samples should be incubated for a time period of from between about 18 to about 24 hours at a temperature of from between about 35° Celsius (C) to about 39° C. Desirably, the inoculated urine sample should be inoculated for a time period of about 20 hours at a temperature of from between about 35° C. to about 39° C. Stock cultures can be maintained according to one's own Compliance Testing Facility (CTF) procedures. Ten micro liters (μl) of each organism should be transferred to a tube of Tryptic Soy Broth (TSB) (10 ml) and incubated for a time period of from between about 18 to about 24 hours at a temperature of from between about 35° C. to about 39° C. The 10 micro liters of each organism should be transferred each day to a fresh tube of TSB. At least 3 passages, but no more that 5 passages, in broth will be used for testing. On the test day, each tube of test organisms will be centrifuged for 15 minutes and the liquid decanted. The pellet will be re-suspended in 25 ml of filtered urine. The test fluid is prepared by adding equal volumes of each re-suspended culture in the filtered urine. The final or total concentration should be between about 4×10⁴to about 4×10⁹ CFU/ml. Desirably, the final or total concentration should be between about 4×10⁵ to about 4×10⁷ CFU/ml. More desirably, the final or total concentration should be approximately 4×10⁶ CFU/ml. Cultures can be kept on ice until the material or products are ready for inoculation for up to about one hour. Bacterial concentrations of each re-suspended culture can be confirmed with viable plate counts.

Glass jars that block the transmittance of light and having polytetrafluoroethylene (PTFE) lined lids, of approximately 500-1,000 ml capacity, should be used to contain the urine-insulted material or product. Each glass jar can be assigned a random number, based on the study randomization. Assignment numbers can be changed for each study. The study sponsor should provide the volume of test fluid (filtered urine inoculated with the known microbes) to insult each material or product. Each material or product will then be placed in a glass jar, protected from light, with an airtight closure. A pipette can be used to insult the material or product and guide the test fluid stream into the center strip of the material or product, thereby allowing the test fluid to be absorbed and/or be wicked into the material or product.

The glass jars with the inoculated urine samples should be capped and stored at a temperature of from between about 35° C. to about 39° C. for a time period of from between about 5.5 to about 6.5 hours. Desirably, the glass jars with the inoculated urine samples will be capped and stored at a temperature of from between about 35° C. to about 39° C. for a time period of between about 5.75 to about 6.25 hours. Most desirably, the glass jars with the inoculated urine samples will be capped and stored at a temperature of from between about 35° C. to about 39° C. for about a time period of about 6.0 hours. The outside of each glass jar should be wiped off with alcohol before they are stored. Separate sets of six (6) replicate inoculated urine samples for each material or product will be prepared for each odor panelist.

The method further includes evaluating the material or product for malodors that smell similar to or the same as urine in order to establish an odor intensity level. Such evaluation is conducted by an odor panelist. Each odor panelist should be a healthy male or female who is at least 18 years of age or older. Each odor panelist should be tested and certified by the test sponsor to make sure they exhibit an exceptional sense of smell. Each odor panelists should also be trained to qualitatively describe overall malodor of the test samples. In addition, each odor panelist should receive basic training, desirably within the last twelve months, from the test sponsor as to how to conduct an odor evaluation and how to report smell findings. Furthermore, each odor panelist should received training in handling hazardous materials and know when, how, where and to whom to report any adverse effects that may occur from conducting such smell tests.

An odor panelist should not perform a smell test if certain events occur. For example, an odor panelist should not perform a smell test if they are not feeling well on the day of the test. Likewise, if the odor panelist is taking prescription or over the counter medication, especially for the treatment of an acute illness, such as upper respiratory problems or an allergy, they should not be perform a smell test. Furthermore, if an odor panelist is experiencing any condition which may impact their sense of smell, such as having a cold, flu or upper respiratory infections, allergy, etc. they should not perform a smell test. Lastly, if the testing could compromise an odor panelist's immune system, then he or she should not participate in a smell test.

The odor panelist will then record his or her odor intensity level and score the odor intensity level using a preselected scale. The scoring or measuring of the odor intensity level can be accomplished by use of a rating system, a ranking system, a magnitude estimation system, or by some other system known to those skilled in the art. By a “rating system” it is meant a system that uses a scale predefined by an experimenter. The rating system can either involve choosing a number from 1 to x (x=any preselected number) or a line scale where the odor panelist makes a mark on a horizontal line of fixed length and the experimenter then measures the distance of the mark from a base point. By a “ranking system” it is meant a system that assigns orders 1 through k, where k is the number of items being compared “simultaneously or at a single testing.” The letter k can range from 2, where the items are difficult to compare, up through a large number when the items are easy to compare (i.e. sorting a deck of cards). By a “magnitude estimation system” it is meant a system that allows the odor panelist to use their own scale and the numeric values are usually touted as ratios. The magnitude estimation system is a more subjective scale wherein each odor panelist determines the odor intensity level of each sample exhibiting a smell similar to urine.

The ranking system allows a direct comparison between items. The rating and magnitude estimation systems use a scale wherein the sample exhibiting the greatest odor is at one end of the scale and the sample exhibiting the least odor is at the opposite end of the scale. For example, the odor intensity level of a sample exhibiting the greatest urine odor can be recorded at the left end of the scale and the odor intensity level of a sample exhibiting the least urine odor can be recorded at the right end of the scale, or vice versa. Odor intensity levels of various samples falling in between are ranked or compared according to the greatest and least urine odor.

The rating and magnitude estimation systems each employ a scale having a certain numerical range. The range can extend from samples having no odor to samples exhibiting an extremely strong urine odor. For example, the rating system can utilize a scale of from 1 to 6, with number 1 representing the greatest odor intensity of urine and number 6 representing the least odor intensity of urine. By “greatest odor intensity” it is meant that a particular sample exhibits a strong urine odor and by “least odor intensity” it is meant that a particular sample exhibits no odor or the least amount of urine odor. Another way of expressing this is to say that the number 1 ranks an odor intensity level identical to urine and number 6 ranks an odor intensity level that does not resemble the smell of urine.

It should be noted that no adverse effects are expected when an odor panelist performs the above-identified smell test. However, if an odor panelist should experience headache, nausea, sore throat, coughing, and/or local irritation such as burning sensation in or around the nose, eyes, or throat, the odor panelist should stop the test and notify his or her test sponsor immediately. In rare cases, an allergic reaction could occur. These types of adverse effects would usually be associated with the upper respiratory tract. A physician should be consulted if any adverse effects persist.

### Evaluation Procedure

One particular evaluation procedure will be explained below. It should be evident to those skilled in the testing art that the actual steps of a test procedure can be varied to suit one's particular needs or requirements. To begin, all specimen containers should remain closed and be incubated at from between about 35° C. to about 39° C. until removed and presented to the odor panelists just prior to the odor evaluations. The odor panelists will be instructed to wash their hands with non-fragranced soap before and after testing. The odor panelist will evaluate each test sample by the following procedure:
1. Secure jar with non-dominant hand and open the lid with the dominant hand.
2. Set the lid down with dominant hand.
3. Lift jar with non-dominant hand to position the jar such that the opening of the jar is about 3 inches under his or her nose.
4. Waft the vapor from the headspace of the jar toward the face with the dominant hand while gently inhaling through his or her nose.

If the odor panelist feels he or she needs a better sniff, he or she may gently sniff the odor in the headspace of the jar by placing his or her nose just above the open jar, and then immediately replace the lid.

The odor panelists can repeat an evaluation after a two minute equilibration period in which the lid has been replaced, if needed. Each odor panelist will evaluate his or her own individual set of samples, each having been incubated for a time period of from between about 5.5 to about 6.5 hours, and record odor intensity. The odor panelists will perform the testing while seated in a testing station. The odor panelists will wear surgical masks between evaluations to cleanse residual odors from his or her nose.

### Scoring Procedure

For the first attribute, all odor panelists will initially be presented with a set of all test samples in their respective containers. The starting arrangement of the material or product samples will correspond to the counter-balanced design. When evaluating a set of samples, each odor panelist will sniff each material or product sample according to the procedure outlined above. As each successive material or product sample is evaluated, it will be returned to its proper order position versus the previously tested material or product samples. The rank orderings of the test samples (from highest to lowest odor intensity compared to the odor of urine) will then be entered onto a paper ballot. This scoring procedure will be repeated for each attribute with delays of two minutes between each set of rankings.

A statistical analysis, such as stratified Cox Proportional Hazards Regression can be used to compare the odor rankings between sample codes. The odor panelist and test session can be used as stratification variables. All comparisons can be made using a 95% Level of Confidence. It should be noted that other statistical methods known to those skilled in statistics can be used, if desired, and the Level of Confidence can be varied at the discretion of the statistician.

It is always a good procedure to have the test sponsor write a final report after the completion of a particular study. The final report can include: a listing of the urine donors, time of donation, quantity of urine collected, the specifics of the test method used, the particular microbes that were inoculated into the test material or product, the actual odor intensity evaluation sheets filled out by each odor panelists, as well as the conclusions relative to the test materials and/or products. It should be known that other information can also be included in the final report.

While the invention has been described in conjunction with several specific embodiments, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

We claim:

1. A method of evaluating and measuring the smell of urine in a material, comprising the steps of:
a) collecting urine samples from at least two donors and pooling said samples together into a combined sample;
b) removing at least about 99% of microbes from said combined sample to obtain a micro-free sample;
c) inoculating said microbe-free sample with at least two known microbes;
d) introducing said inoculated sample into a material and allowing said microbes to grow and emit malodors;
e) evaluating said material for malodors that smell like urine to establish an odor intensity level;
f) recording said odor intensity level; and
g) scoring said odor intensity levels.

2. The method of claim 1 wherein said scoring uses a ranking system of from 1 to 6, with number 1 representing the greatest intensity and number 6 representing the least intensity.

3. The method of claim 1 wherein said scoring uses a rating system which includes a preselected scale that assigns numerical values to represent each odor intensity level.

4. The method of claim 1 wherein said scoring uses a magnitude estimation system which includes an individual subjective scale for each odor intensity level.

5. The method of claim 1 wherein each of said urine samples is tested to determine pH and only those urine samples having a pH of from between 4.5 to 8.0 are pooled.

6. The method of claim 1 wherein said microbe-free sample is inoculated with at least three microbes, two of which are Proteus mirabilis and Enterococcus faecalis.

7. The method of claim 1 wherein said microbe-free sample is inoculated with at least four microbes, three of which are Proteus mirabilis, Enterococcus faecalis and Escherichia coli.

8. The method of claim 7 wherein said microbe-free sample is inoculated with Proteus mirabilis, Enterococcus faecalis, Escherichia coli and Klebsiella pneumoniae.

9. The method of claim 1 wherein said microbe-free sample is inoculated with equal counts of microbes.

10. A method of evaluating and measuring the smell of urine in a material, comprising the steps of:
a) collecting urine samples from at least two donors and pooling said samples together into a combined sample;
b) removing at least about 99% of microbes from said combined sample to obtain a micro-free sample;
c) inoculating said micro-free sample with at least three microbes;
d) introducing said inoculated sample into a material and allowing said microbes to grow and emit malodors;
e) evaluating said material for malodors that smell like urine to establish an odor intensity level;
f) recording said odor intensity level; and
g) scoring said odor intensity levels using a ranking system wherein a low number represents a high odor intensity level and a higher number represents a low odor intensity level.

11. The method of claim 10 wherein said scoring involves a rating system which uses a preselected scale of from 1 to 6 with number 1 representing the greatest odor intensity level and number 6 representing the least odor intensity level.

12. The method of claim 11 wherein said micro-free sample is inoculated with an equal count of microbes.

13. The method of claim 10 wherein said scoring involves a ranking system wherein a high number represents a high odor intensity level and a lower number represents a low odor intensity level.

14. The method of claim 10 wherein said micro-free sample is inoculated with Proteus mirabilis, Enterococcus faecalis, Escherichia coli and Klebsiella pneumoniae.

15. The method of claim 14 wherein said inoculated sample has a total microbe concentration of about 4×10⁶ CFU/ml.

16. A method of evaluating and measuring the smell of urine in a product, comprising the steps of:
a) collecting urine samples from at least two donors and pooling said samples together into a combined sample;
b) removing at least about 99% of microbes from said combined sample to obtain a micro-free sample;
c) inoculating said micro-free sample with at least four microbes, one of which is Proteus mirabilis;
d) introducing said inoculated sample into a product and allowing said microbes to grow and emit malodors;
e) evaluating said product for malodors that smell like urine to establish an odor intensity level;
f) recording said odor intensity level;
g) repeating steps (a-f) for another combined urine sample; and
h) rating said odor intensity level using a preselected scale that employs a 1 to 6 rating system with number 1 representing the greatest odor intensity level and number 6 representing the least odor intensity level.

17. The method of claim 16 wherein said number 1 rates an odor intensity level identical to the smell of urine.

18. The method of claim 16 wherein said number 6 rates an odor intensity level that does not resemble the smell of urine.

19. The method of claim 16 wherein said microbes are allowed to grow in an incubator from about 5.5 to about 6.5 hours at a temperature of from between about 35° C. to about 39° C.

20. The method of claim 16 wherein said inoculated sample has a total microbe concentration of about 4×10⁶ CFU/ml.
